(19) United States
(12) Patent Application Publication
(10) Pub. No.: US 2020/0289473 A1
(43) Pub. Date: Sep. 17, 2020

(54) METHODS OF TREATING NEURODEGENERATION

(71) Applicant: The J David Gladstone Institutes a testamentary trust established under the Will of J David Gladston, San Francisco (CA)

(72) Inventors: Katerina Akassoglou, San Francisco, CA (US); Jae Kyu Ryu, San Francisco, CA (US)

(21) Appl. No.: 16/650,130

(22) PCT Filed: Sep. 25, 2018
(86) PCT No.: PCT/US2018/052694

§ 371 Date: Mar. 24, 2020

Related U.S. Application Data
(60) Provisional application No. 62/562,848, filed on Sep. 25, 2017.
(60) Provisional application No. 62/568,557, filed on Oct. 5, 2017.

(51) Int. Cl. A61K 31/42; A61P 25/28

(52) U.S. Cl. CPC A61K 31/42; A61P 25/28

(57) ABSTRACT

The present invention provides methods and compositions for treating a CNS disease, disorder or injury, (e.g., a CNS demyelinating disease). The present invention provides methods and compositions for preserving or protecting neuroaxonal activity in a subject, preferably a mammalian subject (e.g., a human) by administering one or more compositions that inhibit the activity of γ-glutamyl transpeptidase in the human subject.

### RELATED APPLICATIONS

This International PCT Application claims the benefit of priority to U.S. Provisional Patent Application No. 62/562,848, filed Sep. 25, 2017, and U.S. Provisional Patent Application No. 62/568,557, filed Oct. 5, 2017.

### GOVERNMENT SUPPORT

This invention was made with government support under Grant No. NS052189 awarded by the National Institutes of Health. The government has certain rights in the invention.

### FIELD OF THE INVENTION

This invention relates to compositions and methods of treatment and prevention for diseases, disorders and conditions associated with neurodegeneration.

### BACKGROUND OF THE INVENTION

In the following discussion certain articles and methods will be described for background and introductory purposes. Nothing contained herein is to be construed as an “admission” of prior art. Applicant expressly reserves the right to demonstrate, where appropriate, that the articles and methods referenced herein do not constitute prior art under the applicable statutory provisions.

Neurodegeneration is the progressive loss of structure and/or function of neurons, which may lead to the death of the affected neurons. Neurodegenerative diseases include Alzheimer's disease, Parkinson's disease, amyotrophic lateral sclerosis, Huntington's disease and multiple sclerosis. Although these diseases have different etiologies and symptoms, they all result in progressive degeneration and/or death of neuron cells. Despite their differences, these diseases also display similarities appear that may relate these diseases a cellular or molecular level. Such similarities offer therapeutic advances using modalities common to each of these diseases.

Clinical management of neurodegenerative remains a significant challenge in medicine, however, as they do not address the cellular or molecular basis of the disease. Although some degree of axonal remyelination by oligodendrocytes takes place early during the course of MS, the ability to repair the CNS eventually fails, leading to irreversible tissue injury and an increase in disease-related disabilities. Currently approved therapies for CNS demyelinating diseases, such as multiple sclerosis (MS), are primarily immunomodulatory, and typically do not have direct effects on CNS repair. Similarly, drugs for other neurodegenerative diseases such as Alzheimer's disease and Parkinson's disease do not address the neuronal death and loss of function, but rather ameliorate associated symptoms.

Thus, there is a need for additional therapies that prevent and/or ameliorate neurodegeneration. The present invention meets such need.

### SUMMARY OF THE INVENTION

This Summary is provided to introduce a selection of concepts in a simplified form that are further described below in the Detailed Description. This Summary is not intended to identify key or essential features of the claimed subject matter, nor is it intended to be used to limit the scope of the claimed subject matter. Other features, details, utilities, and advantages of the claimed subject matter will be apparent from the following written Detailed Description including those aspects illustrated in the accompanying drawings and defined in the appended claims.

The present invention provides methods and compositions for treating a CNS disease, disorder or injury that is associated with increased levels of γ-glutamyl transpeptidase. The present invention provides methods and compositions for preserving or protecting neural structure and/or function in a subject in need thereof, preferably in a mammalian subject by administering one or more compositions that inhibit the activity of γ-glutamyl transpeptidase in the subject.

In a primary aspect, the invention provides methods and compositions for treating neurodegeneration in a mammalian subject, preferably a human subject, by administering one or more γ-glutamyl transpeptidase inhibitors in an amount sufficient to reduce one or more symptoms associated with the neurodegeneration in the subject. In specific embodiment, the mammal treated has been diagnosed as having or being at risk for a disease, disorder, or injury associated with neurodegeneration.

In a related aspect, the invention features a method of preventing progression of a CNS disorder in a subject in need of treatment. The method comprises administering to the subject a composition comprising a γ-glutamyl transpeptidase inhibitor in an amount sufficient to thereby arrest the CNS disorder and prevent further neuronal injury and/or death. In certain embodiments, said treatment may result in reduction of one or more symptoms associated with the disease. In some embodiments, the treatment results in reducing, retarding or preventing a relapse, or the worsening of progression of the disease in the subject.

In certain embodiments the present invention provides methods and compositions for preventing or ameliorating demyelination in a subject, preferably a mammalian subject, by administering one or more compositions that inhibit the activity of γ-glutamyl transpeptidase in the human subject.

In other specific embodiments, the present invention provides methods and compositions for enhancing myelination and/or re-myelination in a mammalian subject, preferably a human subject, by administering one or more compositions that inhibit the activity of γ-glutamyl transpeptidase in the human subject.

In still other specific embodiments, the present invention provides methods and compositions for decreasing neurodegeneration associated with plaque formation (e.g., amyloid plaque formation) in a mammalian subject, preferably in a human subject.

In some aspects, the methods and compositions of the invention are used for decreasing neurodegeneration in a patient with multiple sclerosis.

In some aspects, the methods and compositions of the invention are used for decreasing neurodegeneration in a patient with Alzheimer's disease.

In specific aspects, the present invention provides methods and compositions for decreasing neurodegeneration in a mammalian subject with a genetic predisposition for Alzheimer's disease. Examples of such genetic predisposition include mutations in the amyloid precursor protein (APP) gene, Presenilin 1 (PSEN1) and Presenilin 2 (PSEN2) genes, and ApoE4.

In yet another aspect, the invention provides a method for reducing the rate of neurodegeneration in a mammal, comprising administering to the mammal in need thereof an effective amount of a γ-glutamyl transpeptidase inhibitor. In some aspects, the invention provides a method for reducing the rate of demyelination and neuronal injury in a mammal, comprising administering to the mammal in need thereof an effective amount of a γ-glutamyl transpeptidase inhibitor. In other aspects, the invention provides a method of reducing the rate of plaques formation in a mammal, comprising administering to the mammal in need thereof an effective amount of a γ-glutamyl transpeptidase inhibitor.

In yet other aspects, a method for inhibiting microglial activation in the CNS of a mammal with a disease, disorder, or injury involving neurodegeneration, comprising administering to the mammal an effective amount of a composition comprising an inhibitor of γ-glutamyl transpeptidase. In some aspects, the disease is a disease associated with demyelination, dysmyelination. In another aspect, the disease is a neurodegenerative disease associated with the formation of amyloid plaques.

In other embodiments, the invention provides methods for inhibiting microglial activation in CNS neurons comprising contacting CNS neurons with a composition comprising a γ-glutamyl transpeptidase inhibitor.

In certain other embodiments, the mammal treated has developed or is at risk of developing a disease, disorder, or injury involving demyelination or dysmyelination or neuronal injury resulting therefrom. Such diseases, disorders, conditions or injuries include multiple sclerosis (MS), progressive multifocal leukoencephalopathy (PML), encephalomyelitis (EPL), central pontine myelolysis (CPM), adrenoleukodystrophy, Alexander's disease, Pelizaeus Merzbacher disease (PMZ), Wallerian Degeneration, optic neuritis, transverse myelitis, amylotrophic lateral sclerosis (ALS), Huntington's disease, Alzheimer's disease, Parkinson's disease, spinal cord injury, traumatic brain injury, post radiation injury, neurologic complications of chemotherapy, stroke, acute ischemic optic neuropathy, vitamin E deficiency, isolated vitamin E deficiency syndrome, AR, Bassen-Kornzweig syndrome, Marchiafava-Bignami syndrome, metachromatic leukodystrophy, trigeminal neuralgia, acute dissmeminated encephalitis, Guillian-Barre syndrome, Marie-Charcot-Tooth disease and Bell's palsy.

In some embodiments, the present invention provides methods of preventing demyelination and neuronal injury in a mammal in need thereof comprising administering to the mammal a therapeutically effective amount of a pharmaceutical composition comprising a γ-glutamyl transpeptidase inhibitor, wherein the administration of the γ-glutamyl transpeptidase inhibitor prevents an increase in demyelination and injury of CNS neurons in said mammal.

In various embodiments of the invention, the γ-glutamyl transpeptidase inhibitor can be an inhibitor of one or more glutamine-dependent amidotransferases. In more specific embodiments of the invention, the γ-glutamyl transpeptidase inhibitor is a glutamine analog.

In other embodiments of the invention, the γ-glutamyl transpeptidase inhibitor is a small molecule inhibitor. Examples of such γ-glutamyl transpeptidase small molecule inhibitors that can be used in the method of the invention can be found, e.g., in U.S. Pat Appln 20140024685, U.S. Pat Appln 20130085168, and/or U.S. Pat Appln 20100197745. In a specific embodiment, the small molecule is OU749 or an analog or chemical variant thereof.

In one embodiment, the invention provides a method for promoting survival of CNS neurons in a mammal, comprising administering to a mammal in need thereof an effective amount of a composition comprising an inhibitor of γ-glutamyl transpeptidase inhibitor, wherein the mammal treated with said γ-glutamyl transpeptidase inhibitor exhibits decreased neuronal injury in the CNS of said mammal.

The invention also includes pharmaceutical compositions and kits that contain one or more agent that can be used to inhibit degeneration of a neuron or a portion thereof, as described herein. The pharmaceutical compositions and kits can optionally include one or more pharmaceutically acceptable excipients.

In yet another aspect, the invention features a packaged composition (e.g., a packaged pharmaceutical composition) that includes a γ-glutamyl transpeptidase inhibitor that is labeled and/or contains instructions for use of the γ-glutamyl transpeptidase inhibitor for treating a CNS disorder. The γ-glutamyl transpeptidase inhibitor can be in a form suitable for any route of administration, e.g., oral administration, peripheral administration, intrathecal administration, etc. One or more active agents can be included in the packaged pharmaceutical composition.

These aspects and other features and advantages of the invention are described below in more detail. Those skilled in the art will recognize, or be able to ascertain using no more than routine experimentation, many equivalents to the specific embodiments of the invention described herein. Such equivalents are intended to be encompassed by the following claims.

### BRIEF DESCRIPTION OF THE FIGURES

FIG. 1 shows the chemical structure of acivicin.

FIG. 2 is a graph showing the normalized dose response of acivicin on the inhibition of fibrin-induced microglia activation (black line), with no apparent cell toxicity up to 20 μM (grey line).

FIG. 3 is a graph showing the normalized dose response of acivicin on the inhibition of fibrin-induced microglia activation (black line), overlaid with its dose-dependent effect on cell proliferation (grey line).

FIG. 4 is a graph showing the calculated IC₅₀ of acivicin on the inhibition of fibrin-induced (black line) and LPS-induced (grey line) microglia activation.

FIGS. 5A and 5B are graphs showing GGT1 expression in microglia and macrophages in the spinal cord of EAE mice (FIG. 5A) and the cortex of AD mice (FIG. 5B). Data are presented as percentage of GGT1+ area per mouse and presented as mean±s.e.m. (n=5 healthy and n=5 EAE).

FIG. 6 is a graph showing the clinical score for mice injected with the MOG₃₅₋₅₅ peptide induced EAE followed by treatment with saline and acivicin.

FIG. 7 is a graph showing the clinical score for mice injected with the PLP₃₉₋₁₅₁ peptide induced EAE followed by treatment with saline and acivicin.

FIGS. 8A and 8B are graphs showing quantification of Cx3cr1+ microglial accumulation, as assessed by % area of GFP-labeled microglia in the spinal cord (8A), quantification of Ccr2+ monocyte infiltration as assessed by % area of RFP-labeled monocytes (8B).

FIGS. 9A and 9B are graphs showing the quantification of myelin loss (9A) and axonal damage (9B) in acivicin-treated mice compared to saline treated mice.

FIG. 10 is a series of graphs showing pro-inflammatory gene expression in spinal cords at the peak of EAE in acivicin-treated mice compared to saline treated mice

FIG. 11 is a bar graph showing the quantification of GGT-immunoreactivity in human brain tissues from healthy control and multiple sclerosis (MS) patient with active lesions

### DETAILED DESCRIPTION OF THE INVENTION

The practice of the methods and compositions described herein may employ, unless otherwise indicated, conventional techniques of pharmaceutical chemistry, drug formulation techniques, dosage regimes, and biochemistry, all of which are within the skill of those who practice in the art. Such conventional techniques include the use of combinations of therapeutic regimes including but not limited to the methods described herein; technologies for formulations of adjunct therapies used in combination with known, conventional therapies and/or new therapies for the treatment of neurodegeneration, delivery methods that are useful for the compositions of the invention, and the like. Specific illustrations of suitable techniques can be had by reference to the examples herein.

Note that as used herein and in the appended claims, the singular forms “a,” “an,” and “the” include plural referents unless the context clearly dictates otherwise. Thus, for example, reference to “an inhibitor” refers to one or more agents with the ability to inhibit a target molecule, and reference to “the method” includes reference to equivalent steps and methods known to those skilled in the art, and so forth. Unless defined otherwise, all technical and scientific terms used herein have the same meaning as commonly understood by one of ordinary skill in the art to which this invention belongs. All publications mentioned herein are incorporated by reference for the purpose of describing and disclosing devices, formulations and methodologies that may be used in connection with the presently described invention.

Where a range of values is provided, it is understood that each intervening value, between the upper and lower limit of that range and any other stated or intervening value in that stated range is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either both of those included limits are also included in the invention.

In the following description, numerous specific details are set forth to provide a more thorough understanding of the present invention. However, it will be apparent to one of skill in the art that the present invention may be practiced without one or more of these specific details. In other instances, well-known features and procedures well known to those skilled in the art have not been described in order to avoid obscuring the invention.

### Definitions

The terms used herein are intended to have the plain and ordinary meaning as understood by those of ordinary skill in the art. The following definitions are intended to aid the reader in understanding the present invention, but are not intended to vary or otherwise limit the meaning of such terms unless specifically indicated.

A “CNS disorder” can be any disease, disorder or injury associated with the toxicity of a population of cells within the CNS. In one example, the CNS disorder is associated with a pathological process such as neurodegeneration, demyelination, dysmyelination, axonal injury, and/or dysfunction or death of an oligodendrocyte or a neuronal cell, or loss of neuronal synapsis/connectivity. In other examples, the CNS disorder is a disease associated with plaque formation, e.g., amyloid plaque formation. CNS disorders include neurodegenerative disorders that affect the brain or spinal cord of a mammal. In certain embodiments, the CNS disorder has one or more inflammatory components.

The terms “γ-glutamyl transpeptidase” and “GGT” refer to an enzyme capable of hydrolyzing a gamma-glutamylpeptide and/or transferring the gamma-glutamyl radical to other peptides, amino acids, or the like.

The terms “inhibitor of γ-glutamyl transpeptidase” and “γ-glutamyl transpeptidase inhibitor” as used interchangeably herein refer to an agent that inhibits the activity (e.g., signaling activity) or expression of a γ-glutamyl transpeptidase (GGT).

The term “neurodegenerative diseases” includes any disease or condition characterized by problems with movements, such as ataxia, and conditions affecting cognitive abilities (e.g., memory) as well as conditions generally related to all types of dementia. “Neurodegenerative diseases” may be associated with impairment or loss of cognitive abilities, potential loss of cognitive abilities and/or impairment or loss of brain cells. Exemplary “neurodegenerative diseases” include Alzheimer's disease (AD), diffuse Lewy body type of Alzheimer's disease, Parkinson's disease, Down syndrome, progressive multiple sclerosis (MS), dementia, mild cognitive impairment (MCI), amyotrophic lateral sclerosis (ALS), traumatic brain injuries, ischemia, stroke, cerebral ischemic brain damage, ischemic or hemorrhaging stroke, multi-infarct dementia, hereditary cerebral hemorrhage with amyloidosis of the dutch-type, cerebral amyloid angiopathy (including single and recurrent lobar hemorrhages), neurodegeneration induced by viral infection (e.g. AIDS, encephalopathies) and other degenerative dementias, including dementias of mixed vascular and degenerative origin, dementia associated with Parkinson's disease, dementia associated with progressive supranuclear palsy and dementia associated with cortical basal degeneration, epilepsy, seizures, and Huntington's disease.

As used herein, a disease, disorder or condition is “treated” if at least one pathophysiological measurement associated with the disease is decreased and/or progression of a pathophysiological process is reversed, halted or reduced. For example, a disease, disorder or condition can be “treated” if the number of plaques present in the CNS of a patient with a neurodegenerative disease is reduced, remains constant, or the creation of new plaques is slowed by the administration of an agent. In another example, a disease, disorder or condition can treated if one or more symptoms of the disease or disorder is reduced, alleviated, terminated, slowed, or prevented. Measurement of one or more exemplary clinical hallmarks and/or symptoms of a disease can be used to aid in determining the disease status in an individual and the treatment of one or more symptoms associated therewith.

The term “administering” as used herein refers to contacting a neuron or portion thereof with an inhibitor as described herein. This includes administration of the inhibitor to a subject in which the neuron is present, as well as introducing the inhibitor into a medium in which a neuron is cultured. Administration “in combination with” one or more further agents includes concurrent and consecutive administration, in any order.

The term “neuron” as used herein denotes nervous system cells that include a central cell body or soma, and two types of extensions or projections: dendrites, by which, in general, the majority of neuronal signals are conveyed to the cell body, and axons, by which, in general, the majority of neuronal signals are conveyed from the cell body to effector cells, such as target neurons or muscle. Neurons can convey information from tissues and organs into the central nervous system (afferent or sensory neurons) and transmit signals from the central nervous systems to effector cells (efferent or motor neurons). Other neurons, designated interneurons, connect neurons within the central nervous system (the brain and spinal column). Certain specific examples of neuron types that may be subject to treatment according to the invention include cerebellar granule neurons, dorsal root ganglion neurons, and cortical neurons.

The terms “mammal” and “mammalian subject” as used herein refers to any animal classified as a mammal, including humans, higher non-human primates, rodents, and domestic and farm animals, such as cows, horses, dogs, and cats. In preferred embodiments of the invention, the mammal is a human.

The term “pharmaceutical composition” refers to a formulation containing the disclosed compounds in a form suitable for administration to a subject. In a preferred embodiment, the pharmaceutical composition is in bulk or in unit dosage form. The unit dosage form is any of a variety of forms, including, for example, a tablet, capsule, or a vial. The quantity of active ingredient in a unit dose of composition is an effective amount and is varied according to the particular treatment involved.

The phrase “therapeutically effective amount” or “effective amount” used in reference to an agent of the invention is an art-recognized term. In certain embodiments, the term refers to an amount of an agent that produces some desired effect at a reasonable benefit/risk ratio applicable to any medical treatment. In certain embodiments, the term refers to that amount necessary or sufficient to eliminate, reduce or maintain a target of a particular therapeutic regimen. The effective amount may vary depending on such factors as the disease or condition being treated, the particular targeted constructs being administered, the size of the subject or the severity of the disease or condition. One of ordinary skill in the art may empirically determine the effective amount of a particular compound without necessitating undue experimentation.

In certain embodiments, a therapeutically effective amount of an agent for in vivo use will likely depend on a number of factors, including: the rate of release of an agent from a polymer matrix, which will depend in part on the chemical and physical characteristics of the polymer; the identity of the agent; the mode and method of administration; and any other materials incorporated in the polymer matrix in addition to the agent. In certain embodiments, a therapeutically effective amount is the amount effective to induce endogenous oligodendrocyte precursor cell differentiation and/or maturation, thereby promoting myelination in the subject's central nervous system.

### The Invention in General

The present invention is based on the identification of increased levels of γ-glutamyl transpeptidase in the CNS of subjects suffering from neurodegenerative disorders, such as neurodegeneration associated with plaque formation. Administration of an inhibitor of γ-glutamyl transpeptidase to a mammal affected with or at risk of developing neurodegeneration reduces the levels of γ-glutamyl transpeptidase in the CNS of these animals, and the decrease in γ-glutamyl transpeptidase in the CNS of these animals is associated with a decrease in the symptoms and/or progression of the CNS disorder.

As described in further detail below and in the Examples, the methods of the invention are preferably carried out in vivo, although the methods can also be carried out ex vivo in the treatment of nerve grafts or transplants in patients suffering from neurodegenerative disease. In certain examples, the invention provides methods of administration of an inhibitor of γ-glutamyl transpeptidase for treating or preventing degeneration of a central nervous system (CNS) neuron. In various embodiments, the invention provides treating or preventing neurodegeneration associated with demyelination diseases and/or plaque formation in the CNS of a mammalian subject, e.g., multifocal plaques associated with MS or amyloid plaque formation associated with Alzheimer's disease.

### Administration of γ-Glutamyl Transpeptidase Inhibitors

Pharmaceutical formulations of the γ-glutamyl transpeptidase inhibitors described herein are prepared by combining the inhibitor of γ-glutamyl transpeptidase having the desired degree of purity with optional physiologically acceptable carriers, excipients, or stabilizers (see, e.g., Remington's Pharmaceutical Sciences (18^{th} edition), ed. A. Gennaro, 1990, Mack Publishing Co., Easton, Pa.). Acceptable carriers, excipients, or stabilizers are nontoxic to recipients at the dosages and concentrations employed, and can include buffers such as phosphate, citrate, and other organic acids; antioxidants including ascorbic acid, BHA, and BHT; low molecular weight (less than about 10 residues) polypeptides; proteins, such as serum albumin, gelatin or immunoglobulins; hydrophilic polymers such as polyvinylpyrrolidone, amino acids such as glycine, glutamine, asparagine, arginine, or lysine; monosaccharides, disaccharides, and other carbohydrates including glucose, mannose, or dextrins; chelating agents such as EDTA; sugar alcohols such as mannitol or sorbitol; salt-forming counter-ions such as sodium; and/or nonionic surfactants such as Tween, Pluronics, or PEG.

Inhibitors of γ-glutamyl transpeptidase to be used for in vivo administration must be sterile, which can be achieved by filtration through sterile filtration membranes, prior to or following lyophilization and reconstitution. Therapeutic compositions may be placed into a container having a sterile access port, for example, an intravenous solution bag orvial.

The inhibitors of γ-glutamyl transpeptidase can be optionally combined with or administered in concert with each other or other agents known to be useful in the treatment of the relevant disease or condition.

Thus, in the treatment of demyelinating diseases, the inhibitors can be administered in combination with injectable compositions including interferon beta 1a inhibitors or interferon beta 1b inhibitors, glatiramer acetate, and daclizumab; oral medications such as teriflunomide, fingolimod, and dimethyl fumarate; or infused medications such as alemtuzumab, mitoxantrone, or natalizumab.

In the treatment of Alzheimer's disease, inhibitors can be administered with acetylcholinesterase inhibitors (e.g., donepezil, galantamine, and rivastigmine) and/or NMDA receptor antagonists (e.g., memantine).

In the treatment of ALS, for example, inhibitors can be administered in combination with Riluzole (Rilutek), minocycline, insulin-like growth factor 1 (IGF-1), and/or methylcobalamin.

In another example, in the treatment of Parkinson's disease, inhibitors can be administered with L-dopa, dopamine agonists (e.g., bromocriptine, pergolide, pramipexole, ropinirole, cabergoline, apomorphine, and lisuride), dopa decarboxylase inhibitors (e.g., levodopa, benserazide, and carbidopa), and/or MAO-B inhibitors (e.g., selegiline and rasagiline).

The combination therapies can involve concurrent or sequential administration, by the same or different routes, as determined to be appropriate by those of skill in the art. The invention also includes pharmaceutical compositions and kits.

The route of administration of the inhibitors is selected in accordance with known methods, e.g., injection or infusion by intravenous, intraperitoneal, intracerebral, intramuscular, intraocular, intraarterial or intralesional routes, topical administration, or by sustained release systems as described below.

For intracerebral use, the compounds can be administered continuously by infusion into the fluid reservoirs of the CNS, although bolus injection may be acceptable. The inhibitors can be administered into the ventricles of the brain or otherwise introduced into the CNS or spinal fluid. Administration can be performed by use of an indwelling catheter and a continuous administration means such as a pump, or it can be administered by implantation, e.g., intracerebral implantation of a sustained-release vehicle. More specifically, the inhibitors can be injected through chronically implanted cannulas or chronically infused with the help of osmotic minipumps. Subcutaneous pumps are available that deliver proteins through a small tubing to the cerebral ventricles. Highly sophisticated pumps can be refilled through the skin and their delivery rate can be set without surgical intervention. Examples of suitable administration protocols and delivery systems involving a subcutaneous pump device or continuous intracerebroventricular infusion through a totally implanted drug delivery system are those used for the administration of dopamine, dopamine agonists, and cholinergic agonists to Alzheimer's disease patients and animal models for Parkinson's disease, as described by Harbaugh, J. Neural Transm. Suppl. 24:271, 1987; and DeYebenes et al., Mov. Disord. 2:143, 1987.

Suitable examples of sustained release preparations include semipermeable polymer matrices in the form of shaped articles, e.g., films or microcapsules. Sustained release matrices include polyesters, hydrogels, polylactides (U.S. Pat. No. 3,773,919; EP 58,481), copolymers of L-glutamic acid and gamma ethyl-L-glutamate (Sidman et al., Biopolymers 22:547, 1983), poly (2-hydroxyethyl-methacrylate) (Langer et al., J. Biomed. Mater. Res. 15:167, 1981; Langer, Chem. Tech. 12:98, 1982), ethylene vinyl acetate (Langer et al., Id), or poly-D-(−)-3-hydroxybutyric acid (EP 133,988A). Sustained release compositions also include liposomally entrapped compounds, which can be prepared by methods known per se (Epstein et al., Proc. Natl. Acad. Sci. U.S.A. 82:3688, 1985; Hwang et al., Proc. Natl. Acad. Sci. U.S.A. 77:4030, 1980; U.S. Pat. Nos. 4,485,045 and 4,544,545; and EP 102,324A). Ordinarily, the liposomes are of the small (about 200-800 Angstroms) unilamelar type in which the lipid content is greater than about 30 mol % cholesterol, the selected proportion being adjusted for the optimal therapy.

A therapeutically effective amount of a γ-glutamyl transpeptidase inhibitor will depend, for example, upon the therapeutic objectives, the route of administration, and the condition of the patient. Accordingly, it will be necessary for the therapist to titer the dosage and modify the route of administration as required to obtain the optimal therapeutic effect. A typical daily dosage might range from, for example, about 1 μg/kg to up to 100 mg/kg or more (e.g., about 1 μg/kg to 1 mg/kg, about 1 μg/kg to about 5 mg/kg, about 1 mg/kg to 10 mg/kg, about 5 mg/kg to about 200 mg/kg, about 50 mg/kg to about 150 mg/mg, about 100 mg/kg to about 500 mg/kg, about 100 mg/kg to about 400 mg/kg, and about 200 mg/kg to about 400 mg/kg), depending on the factors mentioned above. Typically, the clinician will administer an active inhibitor until a dosage is reached that results in improvement in or, optimally, elimination of, one or more symptoms of the treated disease or condition. The progress of this therapy is easily monitored by conventional assays. One or more agent provided herein may be administered together or at different times (e.g., one agent is administered prior to the administration of a second agent). One or more agent may be administered to a subject using different techniques (e.g., one agent may be administered orally, while a second agent is administered via intramuscular injection or intranasally). One or more agent may be administered such that the one or more agent has a pharmacologic effect in a subject at the same time. Alternatively, one or more agent may be administered, such that the pharmacological activity of the first administered agent is expired prior the administration of one or more secondarily administered agents.

One skilled in the art, upon reading the present specification, will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, intranasal, inhalational, and the like. Dosage forms for the topical or transdermal administration of a compound described herein includes powders, sprays, ointments, pastes, creams, lotions, gels, solutions, patches, nebulized compounds, and inhalants. In a preferred embodiment, the active compound is mixed under sterile conditions with a pharmaceutically acceptable carrier, and with any preservatives, buffers, or propellants that are required.

The present invention also provides a therapeutic kit containing materials useful for the treatment or prevention of the disorders and conditions described above is provided. The therapeutic kit may include a container and a label or package insert on or associated with the container. Suitable containers include, for example, bottles, vials, syringes, etc. The containers may be formed from a variety of materials such as glass or plastic. The container holds a pharmaceutical composition that is by itself or when combined with another agent effective for treating or preventing the condition and may have a sterile access port (e.g., an intravenous solution bag or a vial having a stopper pierceable by a hypodermic injection needle). At least one active agent in the pharmaceutical composition is a γ-glutamyl transpeptidase inhibitor. The label or package insert indicates that the composition is used for treating the condition of choice. Moreover, the kit may include (a) a first container with a pharmaceutical composition contained therein, wherein the composition includes a γ-glutamyl transpeptidase inhibitor; and (b) a second container with a pharmaceutical composition contained therein, wherein the composition includes a different agent. The therapeutic kit in this embodiment of the invention may further include a package insert indicating that the compositions can be used to treat a particular condition. Alternatively, or additionally, the therapeutic kit may further include a second (or third) container including a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes

### Assessment of Treatment

In some aspects, the successful treatment of a subject with a γ-glutamyl transpeptidase is determined by at least a 10%-100% decrease in one or more symptoms of a CNS disorder. Examples of such symptoms include, but are not limited to, slowness of movement, loss of balance, depression, decreased cognitive function, short-term memory loss, long-term memory loss, confusion, changes in personality, language difficulties, loss of sensory perception, sensitivity to touch, numbness in extremities, tremors, ataxia, muscle weakness, muscle paralysis, muscle cramps, muscle spasms, significant changes in eating habits, excessive fear or worry, insomnia, delusions, hallucinations, fatigue, back pain, chest pain, digestive problems, headache, rapid heart rate, dizziness, and visual changes.

For example, clinical signs of MS are routinely classified and standardized, e.g., using an EDSS rating system based on neurological examination and long distance ambulation. As used herein, the “Expanded Disability Status Scale” or “EDSS” is intended to have its customary meaning in the medical practice. EDSS is a rating system that is frequently used for classifying and standardizing MS. The accepted scores range from 0 (normal) to 10 (death due to MS). Typically patients having an EDSS score of about 4-6 will have moderate disability (e.g., limited ability to walk), whereas patients having an EDSS score of about 7 or 8 will have severe disability (e.g., will require a wheelchair). More specifically, EDSS scores in the range of 1-3 refer to an MS patient who is fully ambulatory, but has some signs in one or more functional systems; EDSS scores in the range higher than 3 to 4.5 show moderate to relatively severe disability; an EDSS score of 5 to 5.5 refers to a disability impairing or precluding full daily activities; EDSS scores of 6 to 6.5 refer to an MS patient requiring intermittent to constant, or unilateral to bilateral constant assistance (cane, crutch or brace) to walk; EDSS scores of 7 to 7.5 means that the MS patient is unable to walk beyond five meters even with aid, and is essentially restricted to a wheelchair; EDSS scores of 8 to 8.5 refer to patients that are restricted to bed; and EDSS scores of 9 to 10 mean that the MS patient is confined to bed, and progressively is unable to communicate effectively or eat and swallow, until death due to MS.

In certain embodiments, the evaluation of disease progression includes a measure of upper extremity function (e.g., a 9HP assessment). Alternatively or in combination, disease progression includes a measure of lower extremity function. Alternatively or in combination, disease progression includes a measure of ambulatory function, e.g., short distance ambulatory function (e.g., T25FW). Alternatively or in combination, disease progression includes a measure of ambulatory function, e.g., longer distance ambulatory function (e.g., a 6-minute walk test). In one embodiment, the disease progression includes a measure of ambulatory function other than EDSS ambulatory function. In one embodiment, disease progression includes a measure of upper extremity function e.g., a 9HP assessment, and a measure of ambulatory function, e.g., short distance ambulatory function (e.g., T25FW). In one embodiment, disease progression includes a measure of upper extremity function (e.g., a 9HP assessment) and a measure of lower extremity function. In one embodiment, disease progression includes a measure of upper extremity function (e.g., a 9HP assessment), a measure of lower extremity function, and a measure of ambulatory function, e.g., short distance ambulatory function (e.g., T25FW) and/or longer distance ambulatory function (e.g., a 6-minute timed walk test (e.g., 6MWT)). In one embodiment, one, two or the combination of the T25FW, 6MWT and 9HP assessments can be used to acquire a disease progression value. The measure of ambulatory function (e.g., short distance ambulatory function (e.g., T25FW) or longer distance ambulatory function (e.g., a timed (e.g., 6-minute) walk test (e.g., 6MWT)) and/or measure of upper extremity function (e.g., a 9HP assessment) can further be used in combination with the EDSS to evaluate MS, e.g., progressive forms of MS.

Alzheimer's disease (AD) is a neurodegenerative disorder that results in the loss of cortical neurons, especially in the associative neocortex and hippocampus which in turn leads to slow and progressive loss of cognitive functions, ultimately leading to dementia and death. Major hallmarks of the disease are aggregation and deposition of misfolded proteins such as aggregated beta-amyloid peptide as extracellular senile or neuritic ‘plaques’, and hyperphosphorylated tau protein as intracellular neurofibrillary tangles.

Genetic predispositions for AD are divided into two forms: early-onset familial AD (<60 years), and late-onset sporadic AD (>60 years). Rare, disease causing mutations in Amyloid precursor protein (APP), Presenilin 1 (PSEN1), and Presenilin 2 (PSEN2) genes are known to result in early-onset familial AD while, APOE (allele 4) is the single most important risk factor for late-onset AD. In specific embodiments, the methods of the invention are used to treat subjects with a genetic predisposition for wither early onset familial AD or late-onset sporadic AD.

Although Alzheimer's disease develops differently for every individual, there are many common symptoms. In the early stages, the most common symptom is difficulty in remembering recent events. As the disease advances, symptoms can include confusion, irritability, aggression, mood swings, trouble with language, and long-term memory loss.

Clinical Decision Support Systems (CDSS) comprising computer hardware, software, and/or systems can be used to determine a diagnosis for a patient who has certain symptoms associated with Alzheimer's disease. CDSS often include at least three component parts: a knowledge basis, an inference engine, and a communication mechanism. The knowledge base may comprise compiled information about symptoms, pharmaceuticals, and other medical information. The inference engine may comprise formulas, algorithms, etc. for combining information in the knowledge base with actual patient data. The communication mechanism may be ways to input patient data and to output helpful information based on the knowledge base and inference engine. For example, information may be inputted by a physician using a computer keyboard or tablet and displayed to the physician on a computer monitor or portable device.

In certain aspects, the assessment of treatment includes radiological assessment, e.g., single photon emission computed tomography (SPECT), Positron Emission Tomography (PET), Magnetic Resonance Imaging (MRI) and scintigraphy. For example, multiple sclerosis can be assessed using radiologic assessment of CNS plaques, e.g. by MRI. In another example, AD plaque load can be assessed, e.g., using AP-PET.

The assessment of treatment according to the present invention may also be performed using scanning database systems and methods such as those described in US Appln. No. 20150039346.

### EXAMPLES

The following examples are put forth so as to provide those of ordinary skill in the art with a complete disclosure and description of how to make and use the present invention, and are not intended to limit the scope of what the inventors regard as their invention, nor are the examples intended to represent or imply that the experiments below are all of or the only experiments performed. It will be appreciated by persons skilled in the art that numerous variations and/or modifications may be made to the invention as shown in the specific aspects without departing from the spirit or scope of the invention as broadly described. The present aspects are, therefore, to be considered in all respects as illustrative and not restrictive.

Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees centigrade, and pressure is at or near atmospheric.

### Example I: Demonstration of Acivicin as an Inhibitor of Microglia Activation

To identify inhibitors of microglia activation, an assay as described in U.S. Ser. No. 15/109,163, was used to screen a library of 1907 clinical drugs and bioactive compounds. The criteria for hits were ≥50% inhibition of activation and ≤3% cell death to exclude toxic compounds. This represents a three standard deviation difference from the mean value of negative control activated cells. 128 compounds were selected from the screen that satisfied these criteria for dose response curves. Acivicin, a γ-glutamyl transpeptidase inhibitor, was identified among the compounds and was selected for additional characterization as it was an unexpected target for microglial activation and had not been previously linked with neurological diseases.

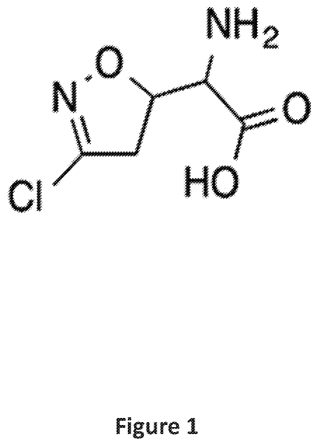

Acivicin is a glutamine analog that irreversibly inhibits glutamine-dependent amidotransferases known to be a potent γ-glutamyl transpeptidase inhibitor (Smith, T. K et al., Proc Natl Acad Sci USA 92, 2360-2364 (1995); Stole, E. The Journal of biological chemistry 269, 21435-21439 (1994). The structure of acivicin is shown in FIG. 1. γ-glutamyl transpeptidase is a plasma membrane ectoenzyme, triggering the catabolism of extracellular glutathione (GSH)⁵. Hanigan, M. H. & Ricketts, W. A. Extracellular glutathione is a source of cysteine for cells that express gamma-glutamyl transpeptidase. Biochemistry 32, 6302-6306 (1993) Acivicin has been used to elucidate aspects of glutathione metabolism and has known anti-tumorigenic activity Poster, D. S. et al., Cancer clinical trials 4, 327-330 (1981); Hidalgo, M. et al. A Phase I and pharmacological study of the glutamine antagonist acivicin with the amino acid solution aminosyn in patients with advanced solid malignancies. Clin Cancer Res 4, 2763-2770 (1998).

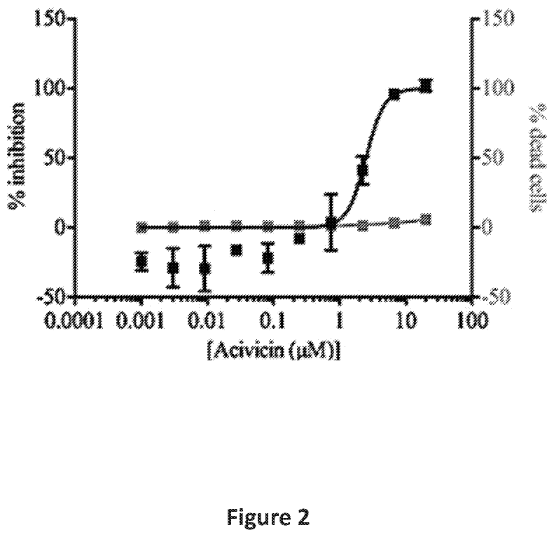

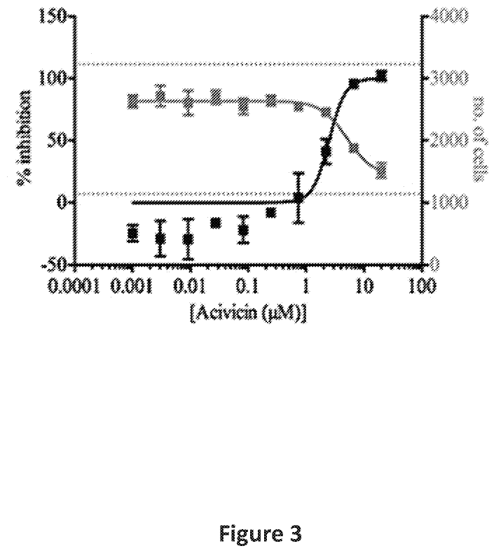

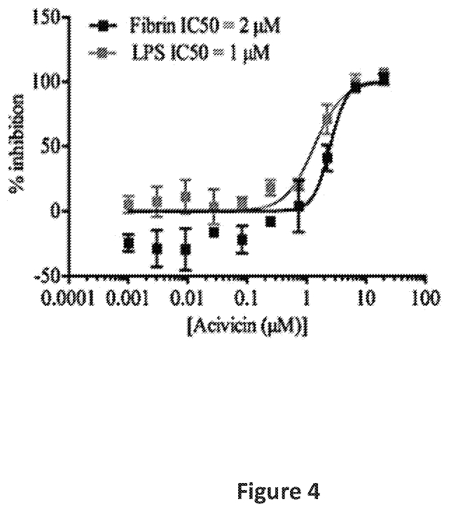

The ability of acivicin to inhibit microglial activation was confirmed, and acivicin exhibited a dose-dependent inhibition of fibrin-induced microglia activation with an IC₅₀ of 2 μM. FIG. 2 shows the normalized dose response of acivicin on the inhibition of fibrin-induced microglia (black line) and toxic cell death was not evident, even at the highest assay concentration of 20 μM (grey line). Throughout assay development, we consistently observed multiple-fold proliferation (increase in cell count) of microglia upon activation. Proliferation of fibrin-activated microglia cells was inhibited by treatment with acivicin. FIG. 3 shows the normalized dose response of acivicin on the inhibition of fibrin-induced microglia activation (black line) overlaid with its dose-dependent effect on cell proliferation (grey line). We also examined the effect of acivicin on microglia activation by other immunostimulatory agents. Acivicin exhibited broad-spectrum inhibition of microglia activation by either fibrin or LPS with comparable low μM potency (FIG. 4).

### Example 2: Gamma-Glutamyl Transpeptidase Expression in Various Neurodegenerative Disease Tissues

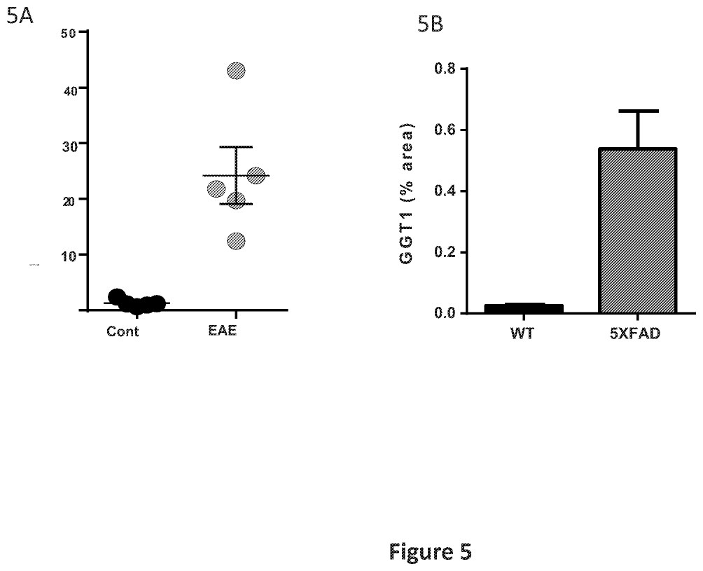

Tissues from various neurodegenerative diseases were tested for γ-glutamyl transpeptidase expression. The difference in expression between γ-glutamyl transpeptidase in normal tissue versus tissues form animal models of MS and AD are shown in FIGS. 5A and 5B.

First, tissues from a mouse model of multiple sclerosis, the EAE model, were tested for γ-glutamyl transpeptidase levels. Spinal cord sections from healthy mice and EAE mice at peak EAE symptoms were immunohistochemically stained for GGT1 in spinal cord sections from healthy mice. A dramatic upregulation of γ-glutamyl transpeptidase was in the spinal cord after EAE. FIG. 5 shows the staining data presented as a mean±s.e.m., with an n=5 for both the healthy mice and the EAE mice. The mice with peak EAE demonstrated a highly elevated level of γ-glutamyl transpeptidase in the spinal cord sections compared to the normal mice.

γ-glutamyl transpeptidase expression was also investigated in brain tissue of 5XFAD mice, which are a transgenic model of Alzheimer's disease. The 5XFAD model over-express human amyloid precursor protein (APP) and presenilin 1 (PS1) harboring five familial AD mutations, which have a high APP expression correlating with a high burden and an accelerated accumulation of the 42 amino acid species of amyloid-β. See, e.g., Oakley H et al., Neurosci. 2006; 26:10129-10140). Cortex sections were prepared from normal mice and 5XFAD mice at 5 months of age, which display the typical amyloid plaques found in advanced Alzheimer's disease. These sections were doubly stained for representative beta-amyloid and γ-glutamyl transpeptidase. γ-glutamyl transpeptidase immunoreactivity was observed around plaques, while no significant staining was observed in the normal controls.

### Example 3: Acivicin Suppression of Disease Progression in an Animal Model of Multiple Sclerosis

To further study the ability of acivicin to inhibit microglial activation, acivicin was next studies for its ability to suppress disease progression and ameliorate the disease symptoms in the EAE animal models of multiple sclerosis. Two models of EAE were used: (1) mice immunized with MOG₃₅₋₅₅ to induce chronic disease and (2) mice immunized with PLP₁₃₉₋₁₅₁ to induce relapsing-remitting EAE.

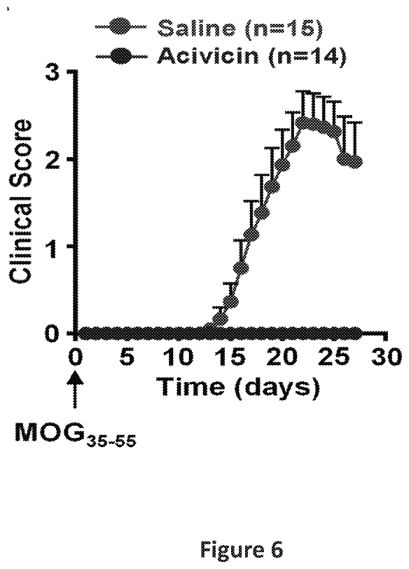

First, to examine the ability of acivicin to prevent disease symptoms and/or progression to multiple sclerosis in a chronic EAE model, acivicin or saline were administered prophylactically to mice immunized with MOG₃₅₋₅₅ to induce chronic disease. The mice were immunized with MOG₃₅₋₅₅ to induce chronic disease. Acivicin or saline was administered daily by intraperitoneal (i.p.) injection, starting 0 day after immunization (n=15 saline, n=14 acivicin). The acute phase of EAE was completely inhibited in MOG₃₅₋₅₅ mice treated with acivicin than in controls treated with vehicle saline (FIG. 6).

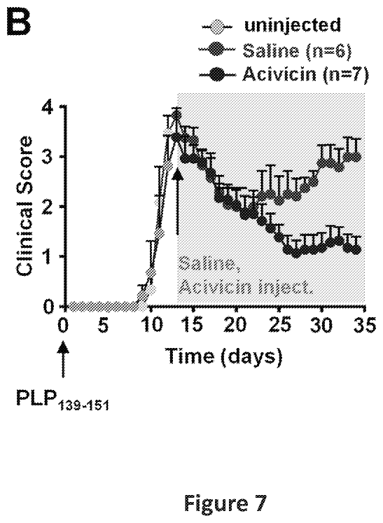

Next, to assess the ability to therapeutic efficacy of acivicin to treat disease symptoms and/or progression in relapsing-remitting (PLP₁₃₉₋₁₅₁) EAE model, mice were administered either acivicin or saline injections daily by intraperitoneal (i.p.) injection starting at the peak EAE phase of the EAE PLP₁₃₉₋₁₅₁. (n=6 saline, and n=7 acivicin). Acivicin decreased the clinical severity of EAE during remission phases (FIG. 7).

### Example 4: Acivicin Suppression of Disease Progression in an Animal Model of Multiple Sclerosis

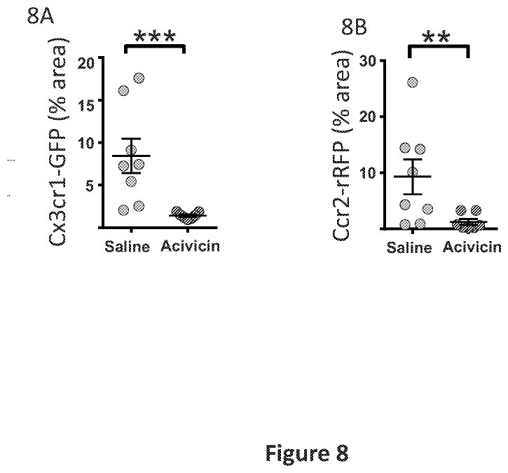

CNS-resident innate immune cell microglia and CNS-infiltrating inflammatory monocytes are essential for the inflammatory progression of EAE. Yamasaki, R. et al. J Exp Med 211, 1533-1549 (2014); Ajami, B. et al, Nature neuroscience 14, 1142-1149 (2011). To determine whether blocking the γ-glutamyl transpeptidase activity with acivicin inhibits microglial activation and infiltration of monocytes/macrophages in vivo in the EAE models, Ccr2^{RFP/+}Cx3cr1^{GFP/+} mice in which resident microglia are GFP-positive and inflammatory monocytes are RFP-positive were used. Ryu, J. K. et al. Nat Commun 6, 8164 (2015). EAE was induced with MOG₃₅₋₅₅, and mice were prophylactically injected with acivicin. Acivicin significantly decreased both the accumulation of Cx3cr1+ microglia (FIG. 8A) and the infiltration of Ccr2+ monocytes (FIG. 8B).

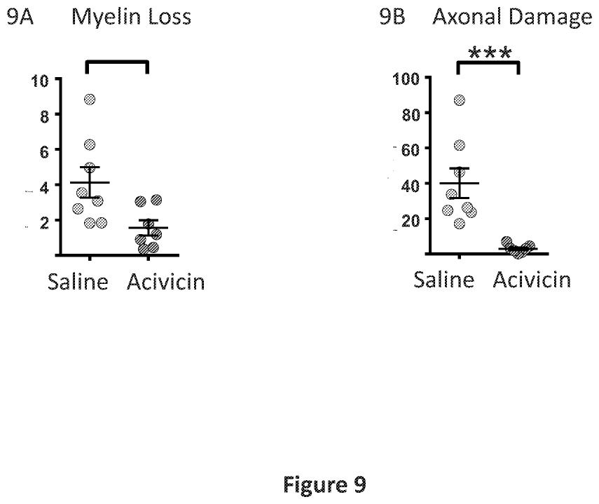

To investigate whether acivicin can protect from myelin damage and neurodegeneration in EAE, MBP and SMI-32 immunohistochemistry was used to assess myelin damage and neuronal injury. Acivicin-treated mice had significant less demyelination and neuronal injury, and showed dramatic reduction of myelin damage (FIG. 9A) and axonal injury (FIG. 9B) in the acivicin-treated mice when compared with saline-treated mice after EAE induction (n=8 saline, n=7 acivicin). These results suggest that the small-molecule acivicin, suppresses microglial activation in vivo, and reduces monocyte/macrophage infiltration and demyelination and neuronal injury.

### Example 5: Blockage of Pro-Inflammatory Gene Expression in Eae Mice Treated with Acivicin

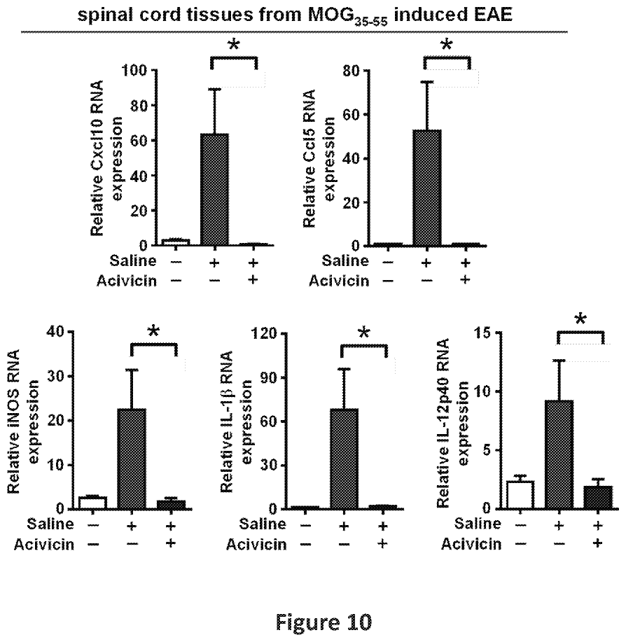

To gain insight into the anti-inflammatory mechanism by which acivicin regulates the progression of inflammation after EAE, real-time PCR analysis was used. Real-time PCR analysis of expression of pro-inflammatory genes in the spinal cords of MOG₃₅₋₅₅ treated mice was performed at the peak of EAE in the saline-treated and acivicin-treated mice (n=7 saline and n=7 acivicin). Gene expression analysis revealed that acivicin inhibits EAE-induced transcription of several genes, including Cxcl10, Ccl5, iNOS, IL-1b, and IL-12p40 that regulate the innate immune response (FIG. 10).

### Example 6: Quantification of GGT-Immunoreactivity in Human Brain Tissues

The amount of γ-glutamyl transpeptidase activity in human brain tissues from healthy control and MS patients with active lesions was determined using the detection method as described, e.g., in Davalos et al., Nature Communications 3, Article number: 1227 (2012). In brief, human brain slices were obtained, cut into 5-μm sections and immunostained with antibodies against γ-glutamyl transpeptidase (1:1,000). Images were acquired with an Axioplan II epifluorescence microscope (Zeiss) equipped with dry Plan-Neofluar objectives (10×0.3 NA, 20×0.5 NA, or 40×0.75 NA). Quantification was performed on thresholded images using ImageJ.

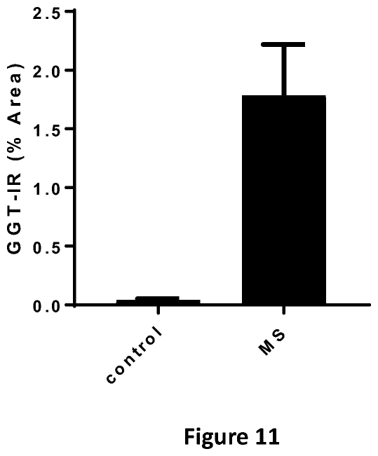

As shown in FIG. 11, the human brain tissues from patients diagnosed with multiple sclerosis show a significant increase in γ-glutamyl transpeptidase as compared to their normal control counterparts. This is consistent with the findings using the mouse model of EAE, in which elevated levels of γ-glutamyl transpeptidase were seen in CNS tissue of the EAE mice (See, e.g., Example 2). This data is also consistent with the predicted efficacy of treating MS in humans using compounds such as acivicin that inhibit γ-glutamyl transpeptidase activity, and suggest that inhibition of γ-glutamyl transpeptidase in humans will protect from myelin damage and neurodegeneration in the CNS of those affected with MS

While this invention is satisfied by aspects in many different forms, as described in detail in connection with preferred aspects of the invention, it is understood that the present disclosure is to be considered as exemplary of the principles of the invention and is not intended to limit the invention to the specific aspects illustrated and described herein. Numerous variations may be made by persons skilled in the art without departure from the spirit of the invention. The scope of the invention will be measured by the appended claims and their equivalents. The abstract and the title are not to be construed as limiting the scope of the present invention, as their purpose is to enable the appropriate authorities, as well as the general public, to quickly determine the general nature of the invention. All references cited herein are incorporated by their entirety for all purposes. In the claims that follow, unless the term “means” is used, none of the features or elements recited therein should be construed as means-plus-function limitations pursuant to 35 U.S.C. § 112, 6.

1. A method of treating or preventing neurodegeneration in a mammal, comprising administering to the mammal an effective amount of a γ-glutamyl transpeptidase inhibitor.

2. The method of claim 1, wherein the γ-glutamyl transpeptidase inhibitor is an inhibitor of one or more glutamine-dependent amidotransferases.

3. The method of claim 1, wherein the γ-glutamyl transpeptidase inhibitor is a glutamine analog.

4. The method of claim 1, wherein said mammal has been diagnosed with a disease, disorder, or injury involving demyelination, dysmyelination, or neurodegeneration.

5. The method of claim 4, wherein said disease, disorder, or injury is selected from the group consisting of multiple sclerosis (MS), progressive multifocal leukoencephalopathy (PML), encephalomyelitis (EPL), central pontine myelolysis (CPM), adrenoleukodystrophy, Alexander's disease, Pelizaeus Merzbacher disease (PMZ), Wallerian Degeneration, optic neuritis, transverse myelitis, amylotrophic lateral sclerosis (ALS), Huntington's disease, Alzheimer's disease, Parkinson's disease, spinal cord injury, traumatic brain injury, post radiation injury, neurologic complications of chemotherapy, stroke, acute ischemic optic neuropathy, vitamin E deficiency, isolated vitamin E deficiency syndrome, AR, Bassen-Kornzweig syndrome, Marchiafava-Bignami syndrome, metachromatic leukodystrophy, trigeminal neuralgia, acute dissmeminated encephalitis, Guillian-Barre syndrome, Marie-Charcot-Tooth disease and Bell's palsy.

6. A method for inhibiting microglial activation and monocyte recruitment in the CNS of a mammal with a disease, disorder, or injury involving demyelination, dysmyelination, or neurodegeneration, comprising administering to the mammal an effective amount of a composition comprising an inhibitor of γ-glutamyl transpeptidase inhibitor

7. The method of claim 6, wherein the γ-glutamyl transpeptidase inhibitor is an inhibitor of one or more glutamine-dependent amidotransferases.

8. The method of claim 6, wherein the γ-glutamyl transpeptidase inhibitor is a glutamine analog.

9. The method of claim 6, wherein said mammal has developed or is at risk of developing diagnosed with a disease, disorder, or injury involving demyelination, dysmyelination, or neurodegeneration.

10. The method of claim 9, wherein said disease, disorder, or injury is selected from the group consisting of multiple sclerosis (MS), progressive multifocal leukoencephalopathy (PML), encephalomyelitis (EPL), central pontine myelolysis (CPM), adrenoleukodystrophy, Alexander's disease, Pelizaeus Merzbacher disease (PMZ), Wallerian Degeneration, optic neuritis, transverse myelitis, amylotrophic lateral sclerosis (ALS), Huntington's disease, Alzheimer's disease, Parkinson's disease, spinal cord injury, traumatic brain injury, post radiation injury, neurologic complications of chemotherapy, stroke, acute ischemic optic neuropathy, vitamin E deficiency, isolated vitamin E deficiency syndrome, AR, Bassen-Kornzweig syndrome, Marchiafava-Bignami syndrome, metachromatic leukodystrophy, trigeminal neuralgia, acute dissmeminated encephalitis, Guillian-Barre syndrome, Marie-Charcot-Tooth disease and Bell's palsy.

11. A method of preventing demyelination and neuronal injury in a mammal in need thereof, the method comprising administering to the mammal a therapeutically effective amount of a pharmaceutical composition comprising a γ-glutamyl transpeptidase inhibitor, wherein the administration of the γ-glutamyl transpeptidase inhibitor prevents an increase in demyelination and injury of CNS neurons in said mammal.

12. The method of claim 11, wherein the γ-glutamyl transpeptidase inhibitor is an inhibitor of one or more glutamine-dependent amidotransferases.

13. The method of claim 11, wherein the γ-glutamyl transpeptidase inhibitor is a glutamine analog.

14. A method for promoting survival of CNS neurons in a mammal, comprising administering to a mammal in need thereof an effective amount of a composition comprising an inhibitor of γ-glutamyl transpeptidase inhibitor, wherein the mammal treated with said γ-glutamyl transpeptidase inhibitor exhibits decreased neuronal injury in the CNS of said mammal.

15. The method of claim 14, wherein the γ-glutamyl transpeptidase inhibitor is an inhibitor of one or more glutamine-dependent amidotransferases.

16. The method of claim 14, wherein the γ-glutamyl transpeptidase inhibitor is a glutamine analog.

17. A method for reducing the rate of demyelination and neuronal injury in a mammal, comprising administering to the mammal an effective amount of a γ-glutamyl transpeptidase inhibitor.

18. The method of claim 17, wherein the γ-glutamyl transpeptidase inhibitor is an inhibitor of one or more glutamine-dependent amidotransferases.

19. The method of claim 17, wherein the γ-glutamyl transpeptidase inhibitor is a glutamine analog.

20. A method for inhibiting microglial activation in CNS neurons comprising contacting CNS neurons with a composition comprising a γ-glutamyl transpeptidase inhibitor.

21. The method of claim 20, wherein the γ-glutamyl transpeptidase inhibitor is an inhibitor of one or more glutamine-dependent amidotransferases.

22. The method of claim 20, wherein the γ-glutamyl transpeptidase inhibitor is a glutamine analog.

23. A method for promoting remyelination in a mammal comprising administering to the mammal an effective amount of a γ-glutamyl transpeptidase inhibitor.

24. The method of claim 23, wherein the γ-glutamyl transpeptidase inhibitor is an inhibitor of one or more glutamine-dependent amidotransferases.

25. The method of claim 23, wherein the γ-glutamyl transpeptidase inhibitor is a glutamine analog.
